(19) United States
(12) Patent Application Publication
(10) Pub. No.: US 2009/0062822 A1
(43) Pub. Date: Mar. 5, 2009

(54) Adaptable clamping mechanism for coupling a spinal fixation element to a bone anchor

(76) Inventors: William J. Frasier, New Bedford, MA (US); Michael Mahoney, Middletown, RI (US); Nicholas Pavento, Marlboro, MA (US); Christopher L. Ramsay, West Wareham, MA (US); David Greg Anderson, Moorestown, NJ (US); Steven Ludwig, Baltimore, MD (US)

Correspondence Address: LAHIVE & COCKFIELD, LLP;FLOOR 30, SUITE 3000, ONE POST OFFICE SQUARE, BOSTON, MA, 02109, US

(21) Appl. No.: 11/897,640

(22) Filed: Aug. 31, 2007

(51) Int. Cl. A61B 17/70; A61B 17/00; A61B 17/56; A61B 17/58

(52) U.S. Cl. 606/151; 606/103

(57) ABSTRACT

An adaptable clamping mechanism for coupling an elongate spinal fixation element to a bone anchor is provided. A seat element and a clamp element of the adaptable clamping mechanism adapt to seat and clamp an elongate spinal fixation element whose longitudinal axis is non-perpendicular relative to the as central axis of the bone anchor. One or both of the seat element and the clamp element may adjust to an out-of-plane orientation of the elongate spinal fixation element by pivoting or rotating in one or more directions. One or both of the seat element and the clamp element may have a deformable portion configured to deform to the orientation of a surface of the elongate spinal fixation element. In addition, the seat element may be configured to provide tactile and/or auditory feedback to a surgeon when the seat element and the elongate spinal fixation element are in contact, facilitating proper positioning of the elongate spinal fixation element in the rod seat when using a minimally invasive rod-first surgical technique.

### FIELD OF THE INVENTION

The present invention relates to a spinal connection device and method for use during orthopedic surgery. More particularly, the present invention relates to an adaptable clamping mechanism that couples an elongate spinal fixation element to a bone anchor.

### BACKGROUND OF THE INVENTION

Spinal fixation systems may be used in surgery to align, adjust and/or fix portions of a spinal column, i.e., vertebrae, in a desired spatial relationship relative to each other. Many spinal fixation systems employ a spinal rod for supporting the spine and for properly positioning components of the spine for various treatment purposes. Vertebral bone anchors, comprising pins, bolts, screws, and hooks, engage the vertebrae and connect the supporting spinal rod to different vertebrae. Spinal fixation elements can be anchored to specific portions of the vertebra. Since each vertebra varies in shape and size, a variety of anchoring devices have been developed to facilitate engagement of a particular portion of the bone.

Pedicle screw assemblies, for example, have a shape and size that is configured to engage pedicle bone. Such screws typically include a threaded shank that is adapted to be threaded into a vertebra, and a head portion having a spinal fixation element-receiving portion, which, in spinal rod applications, is usually in the form of a U-shaped slot formed in the head portion for receiving the rod. A set-screw, plug, cap or similar type of closure mechanism is used to lock the rod into the rod-receiving portion of the pedicle screw.

In conventional spinal surgery, first, anchoring devices are attached to vertebra, then a spinal rod is aligned with the anchoring devices and secured. For example, for conventional pedicle screw assemblies, first the engagement portion of each pedicle screw is threaded into a vertebra. Once the pedicle screw assembly is properly positioned, a spinal fixation rod is seated in the rod-receiving portion of each pedicle screw head. The rod is locked into place by tightening a cap or similar type of closure mechanism to securely interconnect each pedicle screw to the fixation rod. This type of conventional spinal surgical technique usually involves making a surgical access opening in the back of the patient that is almost as long as the length of the spinal rod to be implanted. Because exact placement of the screw assemblies depends on a patient's particular bone structure and bone quality, the exact position of all screw assemblies cannot be known until after all the assemblies are positioned. Adjustments, such as bending, are made to the spinal rod to ensure that it aligns with each screw assembly.

Recently, the trend in spinal surgery has been moving toward providing minimally invasive surgical (MIS) devices and methods for implanting spinal fixation devices. An example of a minimally invasive method is a rod-first method that includes inserting a spinal rod through a first incision and positioning the spinal rod along a patient's spinal column adjacent to one or more vertebra. After the spinal rod is inserted, one or more spinal bone anchors are inserted adjacent to the spinal rod, each through a separate incision. After a spinal bone anchor is inserted and anchored in bone it is coupled to the spinal rod. The rod-first method is a minimally invasive technique in which the bone anchors are inserted adjacent to the rod, after rod insertion, then coupled with the rod, as opposed to a conventional surgical technique in which the anchors are inserted first, then the rod is placed such that it lies over the anchors.

A traditional coupling mechanism for coupling a spinal rod to a bone anchor includes a rod seat disposed on a head of the bone anchor designed to seat a rod whose axis lies in a plane that is perpendicular to a longitudinal axis of the bone anchor, the rod may be said to have in-plane orientation with respect to the bone anchor. However, the spinal rod axis at a particular point along the length of the spinal rod may not lie in a plane perpendicular to the bone anchor axis of the adjacent bone anchor. Instead the spinal rod may have out-of-plane orientation with respect to the bone anchor axis. An adaptable coupling mechanism that can adapt to different out-of-plane rod orientations is needed to properly couple a bone anchor to an adjacent spinal rod that may have out-of-plane orientation relative to the bone anchor. This need is more acute in a rod-first system because the minimally invasive nature of the technique greatly limits the ability to perform rod orientation adjustments, like bending the rod, after the rod is inserted.

### SUMMARY OF THE INVENTION

Embodiments of the present invention provide an adaptable clamping mechanism for coupling an elongate spinal fixation element with a bone anchor and a method of use in surgery. The adaptive clamping mechanism adapts to different non-perpendicular relative orientations between a longitudinal axis of the elongate spinal fixation element and a longitudinal axis of the bone anchor. Embodiments of the adaptable clamping mechanism include a seat element disposed on the bone anchor. The seat element is configured to adapt to seat an elongate spinal fixation element with a longitudinal axis that is non-perpendicular relative to a central axis of the bone anchor. The seat element is also configured to receive the elongate spinal fixation element in an engagement direction that is perpendicular to the longitudinal axis of the spinal fixation element and toward the central axis of the bone anchor. Embodiments of the adaptable clamping mechanism also include a clamp element configured to adapt to hold the elongate spinal fixation element against the seat element. The adaptable clamp mechanism is configured for side engagement with the elongate spinal fixation element further facilitating use with a rod-first system.

In accordance with one exemplary embodiment, the seat element and the clamp element may be configured to adapt over a predetermined minimum angular range of at least ± about 25 degrees relative to an in-plane orientation, which is a total minimum range of about 50 degrees. The adaptable clamping mechanism may further include a securing element configured to secure the clamp element against the elongate spinal fixation element.

According to aspects of the present invention, the seat element may be configured to pivot relative to the central axis of the bone anchor in at least one direction. The seat element may include a pivotable contact disposed on a bottom side of the seat element configured to contact a shaft of the bone anchor in a pivotable fashion. The seat element may include a channel allowing the shaft to pass through the seat element, wherein a channel diameter is significantly larger than a shaft diameter to allow the seat element to pivot relative to the shaft. According to another aspect of the present invention, a portion of the seat element may be formed of a sufficiently compliant material to conform to a surface of the elongate spinal fixation element.

According to other aspects of the present invention, the clamp element may be configured to pivot relative to the bone anchor axis in at least one direction. The clamp element may include a top side and a bottom side, a channel through the clamp element for passing the shaft through. A channel diameter may be sufficiently larger than a shaft diameter to allow the clamp to pivot relative to the shaft. The clamp element may also include a trough recess in the bottom side of the clamp element configured to contact the elongate spinal fixation element.

In accordance with another exemplary embodiment, a portion of the clamp element may be configured to rotate about an axis perpendicular to the bone anchor axis. The clamp element may include an attachment element having a channel for a shaft of the bone anchor to pass through and a rotating element rotatably coupled with the attachment element. The rotating element may be configured to clamp the elongate spinal fixation element against the seat element, and configured to rotate relative to the attachment element about an axis perpendicular to the bone anchor axis.

In accordance with another aspect of the present invention, a bone anchor assembly for securing a spinal fixation element is provided. The bone anchor assembly includes a bone anchor with a central axis and a seat element disposed on the bone anchor. The seat element is configured to adapt to seat an elongate spinal fixation element with a longitudinal axis that is non-perpendicular relative to the central axis of the bone anchor. The seat element is also configured to receive the elongate spinal fixation element in an engagement direction that is perpendicular to the longitudinal axis of the spinal fixation element and toward the central axis of the bone anchor. The bone anchor assembly also includes a clamp element configured to adapt to hold the elongate spinal fixation element against the seat element. The bone anchor assembly further includes a securing element configured to secure the clamp element against the spinal fixation element.

In accordance with another aspect of the present invention, a method is provided for coupling an elongate spinal fixation element that has been positioned to extend along a patient's spinal column to a vertebra of the patient. The method includes providing an adaptable clamping mechanism and implanting a bone anchor and the attached seat element of the adaptable clamping mechanism. The method also includes engaging the elongate spinal fixation element in the seat element from the side and adapting the seat element to an orientation of the elongate spinal fixation element. The method further includes positioning the clamp element of the adaptable clamping mechanism in contact with the elongate spinal fixation element and adapting the clamp element to an orientation of the elongate spinal fixation element. The method also includes securing the elongate spinal fixation element between the clamp element and the seat element.

According to aspects of the present invention, the method may be repeated for coupling the elongate spinal fixation element to an additional bone anchor inserted in one of the patient's vertebrae. Implanting a bone anchor having a shaft and the attached seat element of the adaptable clamping mechanism may include making physical contact between the seat element and the elongate spinal fixation element during insertion of the bone anchor producing feedback to a surgeon regarding a position of the seat element relative to the elongate spinal fixation element.

According to other aspects of the present invention, adapting the seat element to an orientation of the elongate spinal fixation element may include pivoting the seat element relative to the bone anchor. Adapting the seat element to an orientation of the elongate spinal fixation element may include deforming a portion of the seat element to conform to a surface of the elongate spinal fixation element. According to further aspects of the present invention, adapting the clamp element to an orientation of the elongate spinal fixation element may include pivoting the seat element relative to the bone anchor. Adapting the clamp element to an orientation of the elongate spinal fixation element may include rotating a portion of the clamp element about an axis perpendicular to the central axis of the bone anchor.

### BRIEF DESCRIPTION OF THE FIGURES

These and other features and advantages of the mechanisms and methods disclosed herein will be more fully understood by reference to the following detailed description in conjunction with the attached drawings in which like reference numerals refer to like elements through the different views. The drawings illustrate principles of the instruments and methods disclosed herein and, although not to scale, show relative dimensions.

FIG. 1A illustrates a front view of an exemplary embodiment of an adaptable clamping mechanism, having a polyaxial seat element and a polyaxial clamp element, being used to secure an elongate spinal fixation element to a bone anchor, according to aspects of the present invention;

FIG. 1B illustrates a back view of the adaptable clamping mechanism depicted in FIG. 1A;

FIG. 2A illustrates a side view of the adaptable clamping mechanism depicted in FIGS. 1A and 1B;

FIG. 2B illustrates a top perspective view of the adaptable clamping mechanism depicted in FIGS. 1A-2A;

FIG. 3A illustrates a side view of the polyaxial seat element before it is positioned on the bone anchor, according to aspects of the present invention;

FIG. 3B illustrates a top perspective view of the polyaxial seat element before it is positioned on the bone anchor;

FIG. 4 illustrates a vertical cross-section of the adaptable clamping mechanism depicted in FIGS. 1A-2B showing the pivoting contacts of the polyaxial seat element and the polyaxial clamp element;

FIG. 5A illustrates relative orientations of a central axis of the bone anchor, a plane perpendicular to the central axis of the bone anchor, a longitudinal axis of the elongate spinal fixation element and a direction of displacement;

FIG. 5B illustrates a perspective view of the central axis of the bone anchor, the plane perpendicular to the central axis of the bone anchor, a longitudinal axis of the elongate spinal fixation element and a direction of displacement, depicted in FIG. 5A;

FIGS. 6A-6F illustrate various successive stages of side engagement with the elongate spinal fixation element as the bone anchor is inserted into bone, according to aspects of the present invention;

FIG. 6G illustrates the relative positions of the elongate spinal fixation element, the bone anchor, the polyaxial seat, the clamping element and the securing element after the bone anchor has been inserted, the clamping element has been positioned and the clamping mechanism secured, according to aspects of the present invention;

FIG. 7 illustrates a perspective view of another exemplary embodiment of an adaptable clamping mechanism having a seat element with a deformable portion, being used to couple an elongate spinal fixation element with a bone anchor, according to further aspects of the present invention;

FIG. 8A illustrates a perspective view of the adaptable clamping mechanism depicted in FIG. 7 without the elongate spinal fixation element and before the clamping element is positioned;

FIG. 8B illustrates an enlarged top perspective view of the adaptable clamping mechanism depicted in FIGS. 7 and 8A;

FIG. 9A illustrates a front view of another exemplary embodiment of an adaptable clamping mechanism having a polyaxial seat element and a clamp element with a rotating portion, being used to secure an elongate spinal fixation element to a bone anchor, according to other aspects of the present invention;

FIG. 9B is a perspective view of the adaptable clamping mechanism depicted in FIG. 9A;

FIG. 10 is an exploded perspective view of the clamp element of the adaptable clamping mechanism depicted in FIGS. 9A and 9B; and

FIG. 11 illustrates a flow diagram for an exemplary embodiment of a method of using an adaptable clamping mechanism of the invention.

### DETAILED DESCRIPTION OF THE INVENTION

Certain exemplary embodiments will now be described to provide an overall understanding of the principles of the structure, function, manufacture, and use of the adaptable clamping mechanisms, bone anchor assemblies that include adaptable clamping mechanisms and methods disclosed herein. Examples of these embodiments are illustrated in the accompanying drawings. Those of ordinary skill in the art will understand that the adaptable clamping mechanisms and methods of use specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Exemplary embodiments described herein concern an adaptable clamping mechanism for coupling an elongate spinal fixation element (such as a spinal rod) to a bone anchor (such as a pedicle screw). An exemplary embodiment of an adaptable clamping mechanism includes a seat element disposed on the bone anchor and configured to adapt to seat an elongate spinal fixation element with a longitudinal axis that is non-perpendicular relative to a central axis of the bone anchor. The seat element is also configured to receive the elongate spinal fixation element in an engagement direction that is perpendicular to the longitudinal axis of the elongate spinal fixation element and toward the central axis of the bone anchor, which is referred to as substantially side engagement herein. An exemplary embodiment of an adaptable clamping mechanism also includes an adaptable clamp element configured to adapt to hold the elongate spinal fixation element against the seat element. The adaptable clamp mechanism is configured for substantially side engagement with the elongate spinal fixation element. Substantially side engagement is positioning of the elongate spinal fixation element on the seat element by displacing at least a portion of the elongate spinal fixation element in a direction substantially perpendicular to both the elongate spinal fixation element axis and the bone anchor axis. Substantially side engagement facilitates coupling between a rod and a bone anchor in a “rod first” minimally invasive technique in which bone anchors are inserted adjacent to a rod. Rod first techniques and other minimally invasive techniques make manipulation of a rod, such as bending, after insertion very difficult. Adaptable clamping mechanisms reduce the need for rod manipulation by coupling a rod to a bone anchor with a non-perpendicular orientation relative to each other. Additionally, a combination of the seat element and a flared portion of the bone anchor shaft may provide feedback regarding a position of the elongate spinal fixation element relative to the seat element.

An elongate spinal fixation element, such as a spinal rod, may be bent or curved in shape. For an elongate spinal fixation element that is not straight, the direction of a longitudinal axis of the elongate spinal fixation element changes at different positions along a length of the elongate spinal fixation element, because it is curved. As used herein, the longitudinal axis of an elongate spinal fixation element, also called the elongate spinal fixation element axis, refers to the direction of the longitudinal axis of the elongate spinal fixation element at a position along the length of the elongate spinal fixation element that is closest to the adjustable clamping mechanism.

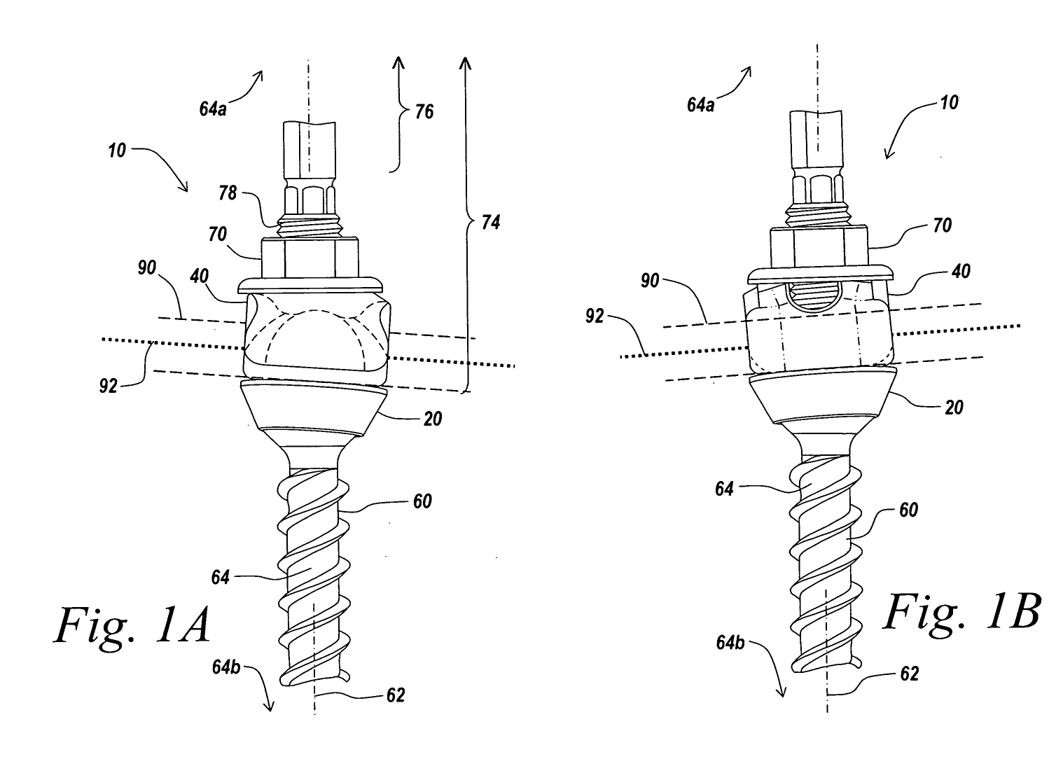

FIGS. 1A and 1B illustrate an exemplary embodiment of an adaptable clamping mechanism 10 for coupling an elongate spinal fixation element 90 to a bone anchor 60. The elongate spinal fixation element 90 has a longitudinal axis 92. The bone anchor 60 has a central axis 62. If the longitudinal axis 92 of the elongate spinal fixation element 90 is not perpendicular to the central axis 62 of the bone anchor 60, then the spinal fixation element 90 is said to have an out-of-plane orientation relative to the bone anchor. If the longitudinal axis 92 of the elongate spinal fixation element 90 is perpendicular to the central axis 62 of the bone anchor 60 then the elongate spinal fixation element 90 is said to have an in-plane orientation relative to the bone anchor 60. The bone anchor 60 has shaft 64 with a proximal end 64a and a distal end 64 for engaging bone. A surface or a side that substantially faces the proximal end 64a of the shaft 64 will be referred to as a top surface, and a surface or a side that substantially faces the distal end 64b of the shaft 64 will be referred to as a bottom surface.

As depicted in FIGS. 1A and 1B, the adaptable clamping mechanism 10 includes a seat element 20 disposed on the bone anchor 60 and configured to adapt to seat the elongate spinal fixation element 90 having a longitudinal axis 92 that is non-perpendicular with respect to the central axis 62 of the bone anchor 60 (the spinal fixation element 90 has out-of-plane orientation relative to the bone anchor 60). The seat element 20 is configured to seat an elongate spinal fixation element with in-plane orientation relative to the bone anchor 60, as well as the elongate spinal fixation element 90 with out-of-plane orientation with respect to the bone anchor 60.

The adaptable clamping mechanism 10 also includes a clamp element 40 configured to adapt to hold the elongate spinal fixation element 90, with out-of-plane orientation relative to the bone anchor 60, against the seat element 20. Like the seat element 20, the clamp element 40 is configured to clamp an elongate spinal fixation element with in-plane orientation, as well the elongate spinal fixation element 90 with out-of-plane orientation.

According to aspects of the present invention, the seat element 20 of the exemplary adaptable clamping mechanism 10 may pivot in one or more directions relative to the bone anchor 60 to adapt to the out-of-plane orientation of the elongate spinal fixation element 90. The seat element 20 of the adaptable clamping mechanism 10 pivots in more than one direction making it a polyaxial seat element. Similarly, the clamping element 40 may pivot relative to the bone anchor 60 in one or more direction to adapt to the out-of-plane orientation of the elongate spinal fixation element 90. According to other aspects of an exemplary embodiment, the adaptable clamping mechanism 10 may further include a securing element 70 that holds the clamping element 40 against the elongate spinal fixation element 90. Alternately a securing mechanism may be included in the clamp element 40. The securing element 70 may engage threads 73 on the shaft 64. Tightening the securing element 70 against the clamping element 40 secures and locks the elongate spinal element 90 within the adaptable clamping mechanism 10.

According to other aspects of the present invention, the shaft 64 may include an extension portion 74 that extends from the location of the seat element 20 to the proximal end 64a of the shaft. The extension portion 74 of the shaft 64 may include a breakaway portion 76 disposed at the proximal end 64a of the shaft 64. The breakaway portion 76 facilitates positioning and inserting the bone anchor 60 with the attached seat element 20 and aids in positioning the clamp element 40. After the elongate spinal fixation element 90 is clamped and secured by the adaptable clamping mechanism 10, the breakaway portion 76 of the shaft may be separated from the rest of the shaft 64 and removed from the patient. The breakaway portion 76 of the shaft 64 may be configured such that a torque force used to tighten the securing element 70 also causes the breakaway portion 76 of the shaft 64 to separate from the rest of the shaft 64. Alternatively, the breakaway portion 76 of the shaft 64 may be separated from the rest of the shaft 64 using a cutting tool.

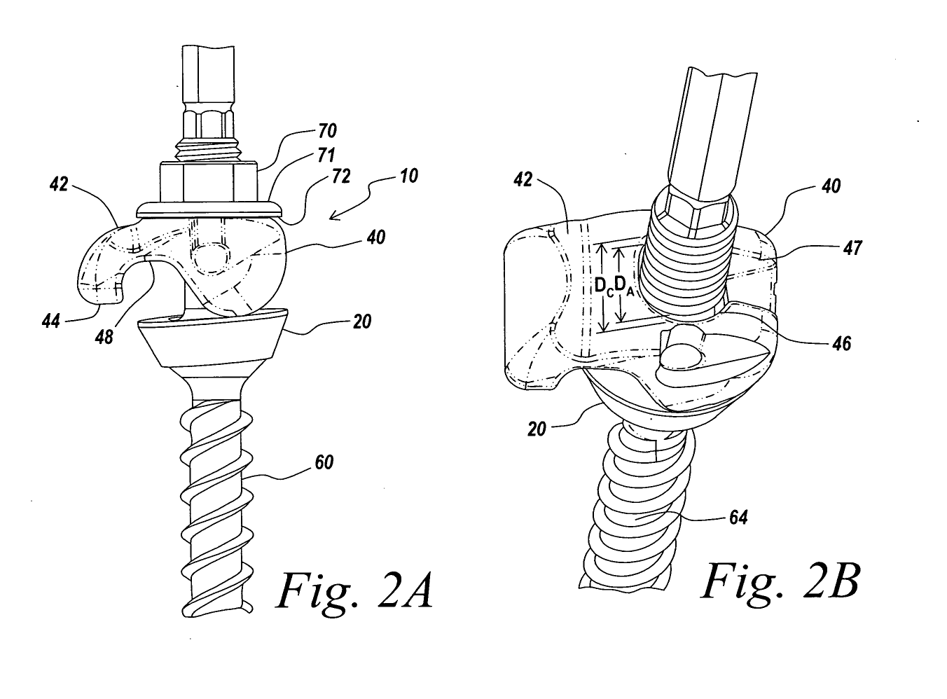

FIGS. 2A and 2B illustrate a side view and a top view of the adaptable clamping mechanism 10. The clamp element 40 has a top side 42 facing the proximal end of the shaft 64a and a bottom side 44 facing the seat element 20. According to aspects of the invention, the clamp element may have a channel 46 for passing the shaft 64 through. The channel 46 of the clamp element 40 may have a channel width D_{C} that is sufficiently larger than a shaft diameter D_{A} to allow the clamp element 40 to pivot relative to the shaft 64. The bottom side 44 of the clamp element 40 may have a trough recess 48 configured to receive the spinal fixation element 90.

According to other aspects of the invention, the clamp element 40 may be sized and dimensioned to be inserted into a patient through a minimally invasive surgical access port, such as a cannula. Additionally, the clamp element 40 may have a slot 47 allowing it to swivel to a position where the trough recess 48 of the clamp element 40 faces the shaft 64. In this position the clamp element 40 has a smaller insertion profile allowing it to be inserted though a smaller minimally invasive surgical access port. Techniques and instruments for minimally invasive insertion of a bone anchor and a connecting element are discussed in detail in the related applications: application DUQ-034 entitled “Minimally Invasive Guide System,” filed on Aug. 31, 2007, and DUQ-037 entitled “Method and System for Securing a Rod to a Bone Anchor with a Connector,” filed on Aug. 31, 2007.

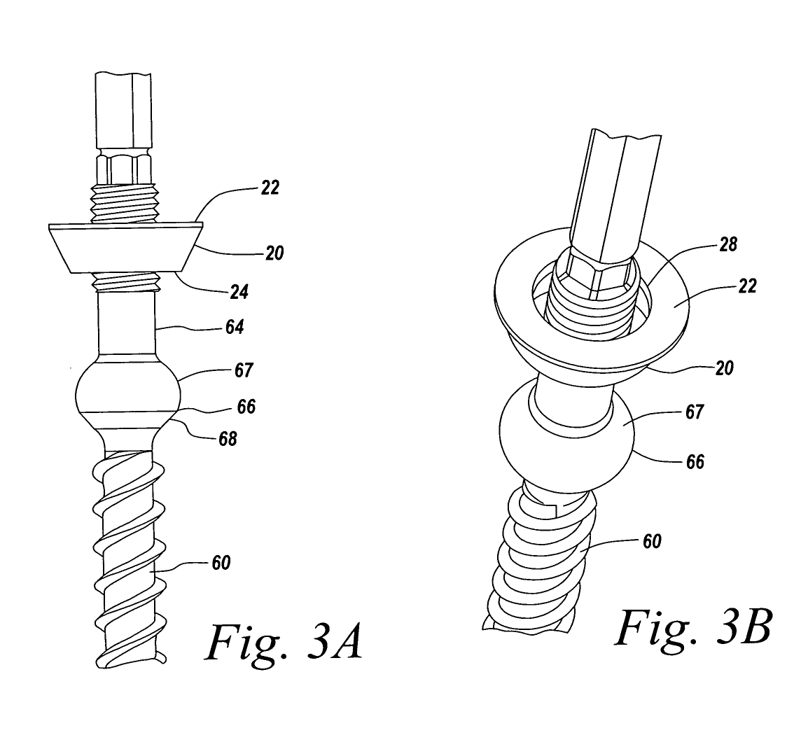

FIGS. 3A and 3B depict the bone anchor 60 and the seat element 20 before the seat element 20 has been positioned on the bone anchor 60. The seat element 20 has a top side 22 facing the clamp element 40 and a bottom side 24 facing the distal end 64b of the shaft. The seat element 20 may include a channel 28 allowing the shaft 64 to pass through the seat element 20.

A shape of a shaft bulge 66 on the shaft 64 and a shape of the bottom side 24 of the seat element 20 may be configured for pivoting in multiple directions (polyaxial pivoting). The shaft bulge 66 may have a convex substantially spherically shaped side 67 that faces the seat element 20, and a flared side 68. The bottom side 24 of the seat element 20 that faces the shaft bulge 66 may have a concave substantially spherical shape configured to pivotably contact the convex substantially spherically shaped side 67 of the shaft bulge 66. Before insertion into a patient, the seat element 20 is pushed in the direction of the distal end of the shaft against the shaft bulge 60, causing the seat element 20 to “snap” onto the shaft bulge 66. An interference fit between the seat element 20 and the shaft bulge 66 prevents the seat element 20 from separating from bone anchor 60 during insertion of the bone anchor 60 into a patient.

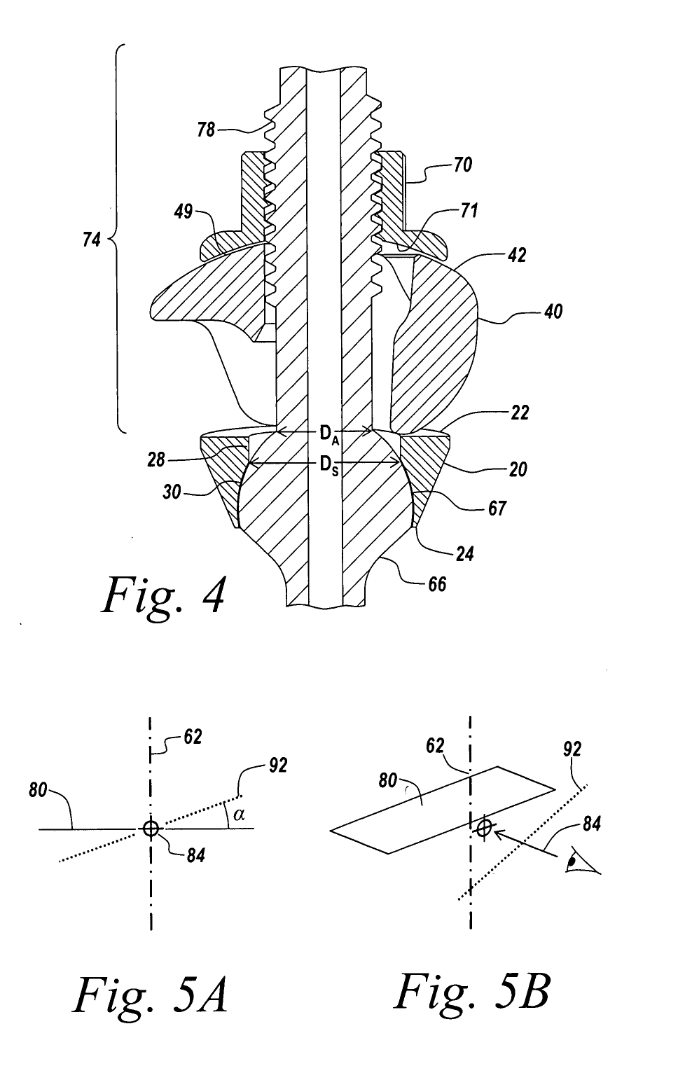

FIG. 4, which depicts a cross-section of the adaptable clamping mechanism, further illustrates a contact area between the seat element 20 and the shaft bulge 66 of the bone anchor 60. The bottom side 24 of the seat element and the top side 67 of the shaft bulge 66 contact each other over a spherically curved area forming polyaxial pivotable contact 30. Due to an interference fit between the seat element 20 and the shaft bulge 60, the seat element 20 pivots on the convex substantially spherically shaped side 67 of the shaft bulge 66, but stays in contact with the shaft bulge 66. The substantially spherical shape of the contact area between the bottom side 24 of the seat element 20 and the convex substantially spherically shaped side 67 of the shaft bulge 66 forms a polyaxial pivotable contact 30. A diameter D_{S} of the channel 28 may be significantly larger than the shaft diameter D_{A} to allow the seat element 20 to pivot relative to the shaft 64.

FIG. 4 further illustrates contact between the securing element 70 and the clamp element 40. The securing element 70 has a first side 71 that faces the clamp element 40. According to aspects of the invention, a shape of the top side 42 of the clamp element 40 may be substantially convex spherical, and a shape of the first side 71 of the securing element 70 may be substantially concave spherical and complementary to the shape of the top side 42 of the clamp element 40. The complementary spherical surfaces ensure that tightening the securing element 70 against the clamp element 40 will create substantially even pressure on the top side 42 of the clamp element 40, regardless of the angle of pivot of the clamp element 40 relative to the securing element 70.

The adaptable clamp mechanism 10 is configured for substantially side engagement with the elongate spinal fixation element 90. Substantially side engagement facilitates coupling of an elongate spinal fixation element 90 with a bone anchor 60 in a minimally invasive surgical (MIS) technique such as a “rod-first” technique. As discussed previously, in a “rod-first” surgical technique the elongate spinal fixation element 90, such as a rod, is positioned in the patient before the bone anchor 60. In a conventional technique the bone anchors are usually inserted first, and then the elongate spinal fixation element is seated in the receiving portion of each bone anchor, often by lowering the elongate spinal fixation element from above into a “U” shaped slot located in the head of the bone anchor. This may be described as substantially vertical engagement. In another conventional technique, the bone anchors are inserted first, and then the elongate spinal fixation element is threaded through an opening in the head of the bone anchor, being displaced in a direction substantially parallel to the longitudinal axis of the elongate spinal fixation element. This may be described as horizontal in-line engagement. Neither vertical engagement nor horizontal in-line engagement is suitable for use in a surgical technique in which the elongate spinal fixation element is inserted and positioned before the bone anchors.

In a rod-first technique, the bone anchors 60 are inserted adjacent to the elongate spinal fixation element 90, as opposed to a conventional technique where the elongate spinal fixation element is placed on top of the heads of the bone anchors or threaded through openings in the heads of the bone anchors. Substantially side engagement allows the bone anchors 60 to couple to the adjacent elongate spinal fixation element 90 with minimal movement or displacement of the spinal fixation element 90.

FIGS. 5A and 5B illustrate the relevant axes, and orientations to describe substantially side engagement. FIG. 5A illustrates relative orientations between the central axis 62 of the bone anchor 60, a plane 80 perpendicular to the central axis 62, and the longitudinal axis 92 of the elongate spinal fixation element 90. Because the longitudinal axis 92 of the elongate spinal fixation element 90 is not perpendicular to the central axis 62 of the bone anchor 60, the longitudinal axis 92 intersects the plane 80, giving rise to the term “out-of-plane” orientation. The angle at which the longitudinal axis 92 of the elongate spinal fixation element 90 intersects the plane 80 is angle α. If the fixation element axis 92 is parallel to the plane 80 then the fixation element axis 92 is said to have an “in-plane” orientation relative to the bone anchor axis 62, and the angle a is zero. A direction for side engagement 84 is perpendicular to the longitudinal axis 92 of the elongate spinal fixation element 90 and toward the central axis 62 of the bone anchor 60. Another way to describe the direction 84 for side engagement is that it perpendicular to both the longitudinal axis 92 of the elongate spinal fixation element 90 and perpendicular to the central axis 62 of the bone anchor 60. FIG. 5B illustrates a perspective view of the relative orientations of the elements depicted in FIG. 5A. Optimally, the seat element and the clamp element may be configured to adapt over a predetermined minimum angular range to accommodate an angle a of at least ± about 25 degrees, which spans a total minimum angular range of about 50 degrees.

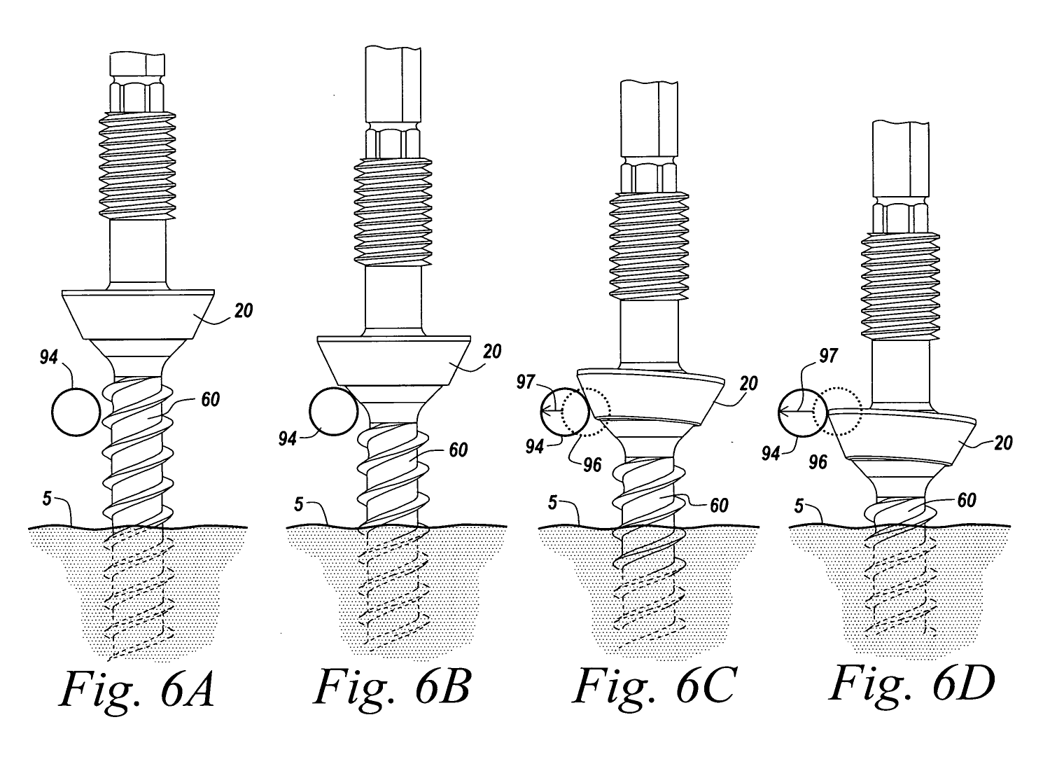

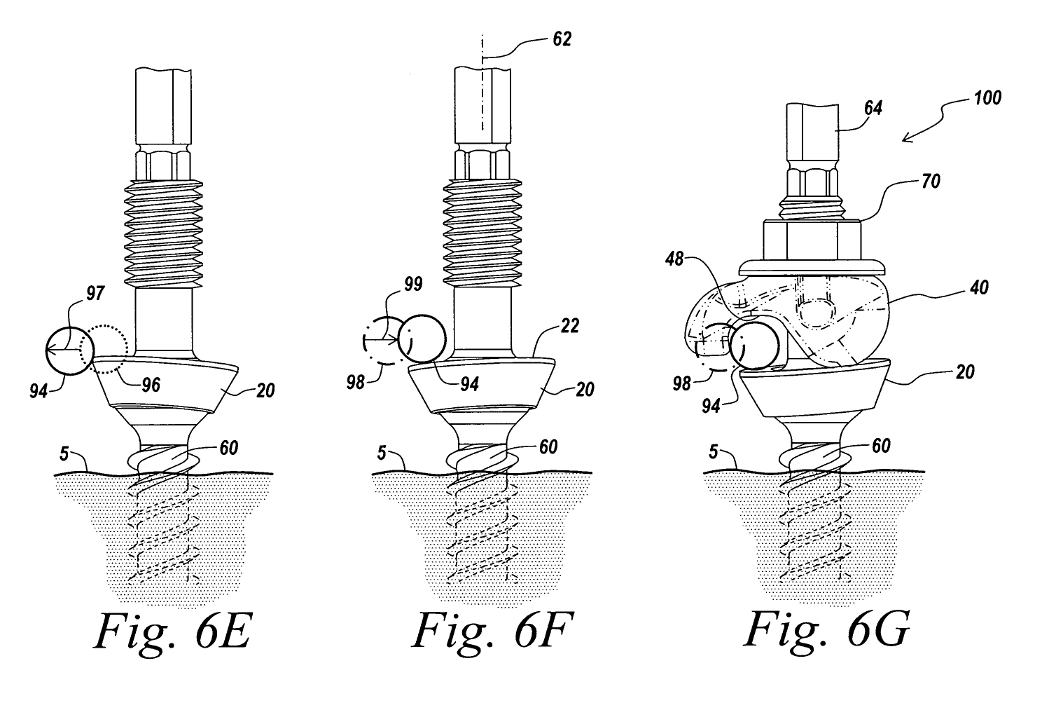

FIGS. 6A-6F illustrate successive various stages of substantially side engagement of the adaptable clamp element mechanism 10 with an elongate spinal fixation element, in this case a rod 94. As illustrated in FIG. 6A, the rod 94 has been inserted into the patient and positioned, and the bone anchor 60 and the attached seat element 20 are being implanted into the patient's bone 5 adjacent to the rod 94. In FIG. 6B the bone anchor 60 and seat element 20 have been inserted deep enough that the bone anchor shaft bulge 66 comes into contact with the rod 94. In FIG. 6C the bone anchor 60 and seat element 20 have been inserted further and contact between the seat element 20 and the rod 94 has displaced the rod 94 away from an axis of the bone anchor 60, with respect to its original position 96, by a distance indicated by arrow 97. In addition, contact with the rod 94 has caused the seat element 20 to pivot away from the rod 94. In FIG. 6D further insertion causes further displacement in a direction 97. In FIG. 6E the bone anchor 60 and seat element 20 have been inserted deeper and the rod 94 is at a point of maximal side displacement.

Further insertion of the bone anchor 60 and seat element 20 causes a top edge of the seat element 20 to move past the rod 94 causing the rod 94 to “snap” from the position of maximal displacement 98 away from the bone anchor 60 to its original position 96, a displacement indicated by arrow 98. This displacement 98 is perpendicular to a longitudinal axis of the rod 94 and toward the central axis 62 of the bone anchor 60. The sudden change in rod 94 position, and the sudden change in force on the bone anchor 60 and the seat element 20, may be felt by a surgeon through instruments connected to the bone anchor 60 providing feedback to the surgeon regarding the position of the rod 94 relative to the seat element 20. Additionally, the sudden change in rod 94 position, and the sudden change in force on the bone anchor 60 and the seat element 20, may produce a sound audible to the surgeon. In this manner the seat element may provide auditory and/or tactile feedback regarding a position of the rod 94 relative to a position of the seat element 20 during insertion of the bone anchor 60. After the rod 94 “snaps” back to its original position, the rod 94 is properly seated in the seat element 20.

When the rod 94 “snaps” into position on the seat element 20, the rod 94 exerts force on the seat element 20 that causes the seat element 20 to pivot until at least a portion of the top side 22 of the seat element is parallel to the longitudinal axis of the rod 94. After the elongate spinal fixation element 90 is seated, the clamp element 40 is moved along the shaft 64 until it is in contact with the rod 94. Due to gravity and/or externally applied forces the clamp element 40 pivots until an axis of the clamp element trough recess 48 is parallel to the rod 90. After the clamp element 40 is positioned, it may be secured by the securing element 70 as illustrated in FIG. 6G.

Another exemplary embodiment, also depicted in FIG. 6G, is a bone anchor assembly 10 for securing an elongate spinal fixation element, such as a rod 94. The bone anchor assembly 10 includes the bone anchor 60, and the seat element 20 disposed on the bone anchor and adapted to seat the rod 94 whose longitudinal axis is non-perpendicular with respect to the central axis 62 of the bone anchor 60. The seat element 20 is also configured to receive the elongate spinal fixation element in an engagement direction that is perpendicular to the longitudinal axis of the rod 94 and toward the central axis 62 of the bone anchor 60. The clamping element 40 is configured to adapt to hold the rod 94 against the seat element 20. The securing element 70 is configured to secure the clamp element 40 against the rod 94. Any other aspects and features of the bone anchor 60, the seat element 20, the clamping element 40, and the securing element 70 discussed herein may be incorporated into the bone anchor assembly 10, according to aspects of the present invention.

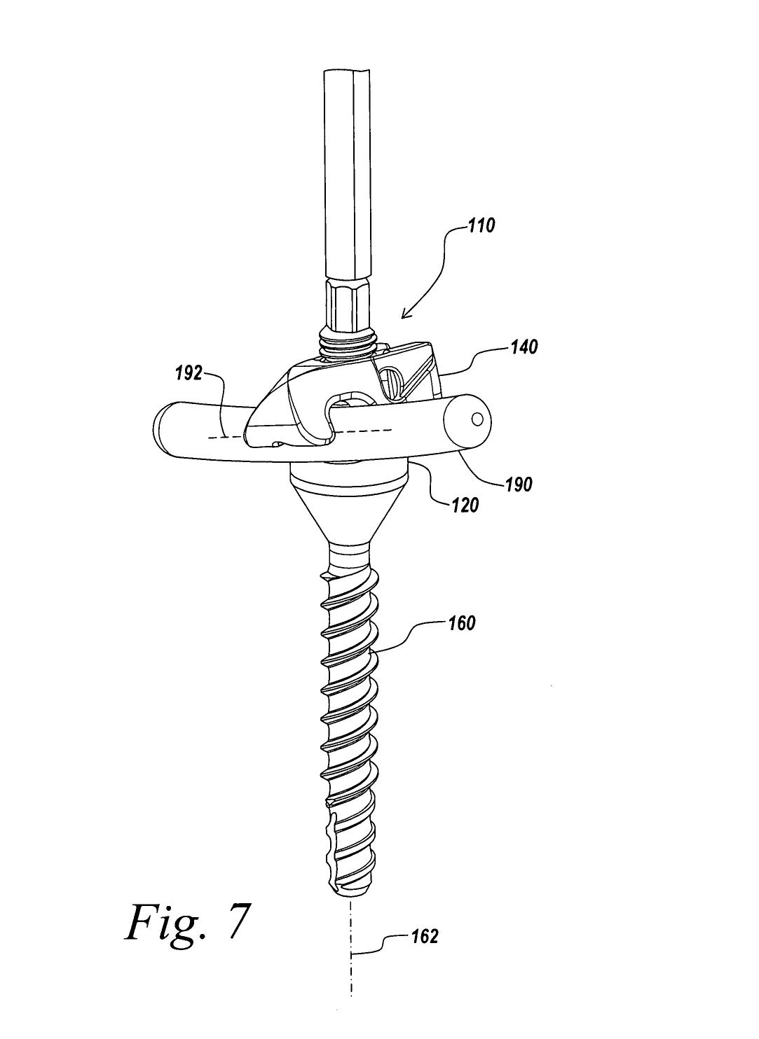

Another exemplary embodiment of an adaptable clamping mechanism 110 for coupling an elongate spinal fixation element 190 to a bone anchor 160 is depicted in FIG. 7. The adaptable clamping mechanism 110 includes a seat element 120 disposed on the bone anchor 160 and configured to adapt to seat a rod 190 with a longitudinal rod axis 192 that is non-perpendicular relative to a central axis 162 of the bone anchor 160. The adaptable clamping mechanism 110 also includes a clamp element 140 configured to adapt to seat the rod 190. The seat element 120 and the clamp element 140 are configured to couple and secure a rod with a perpendicular orientation relative to the central axis 162 of the bone anchor 160, as well being configured to adapt to seat and clamp the rod 190 with a longitudinal axis 192 that is non-perpendicular relative to the central axis 160 of the bone anchor 162.

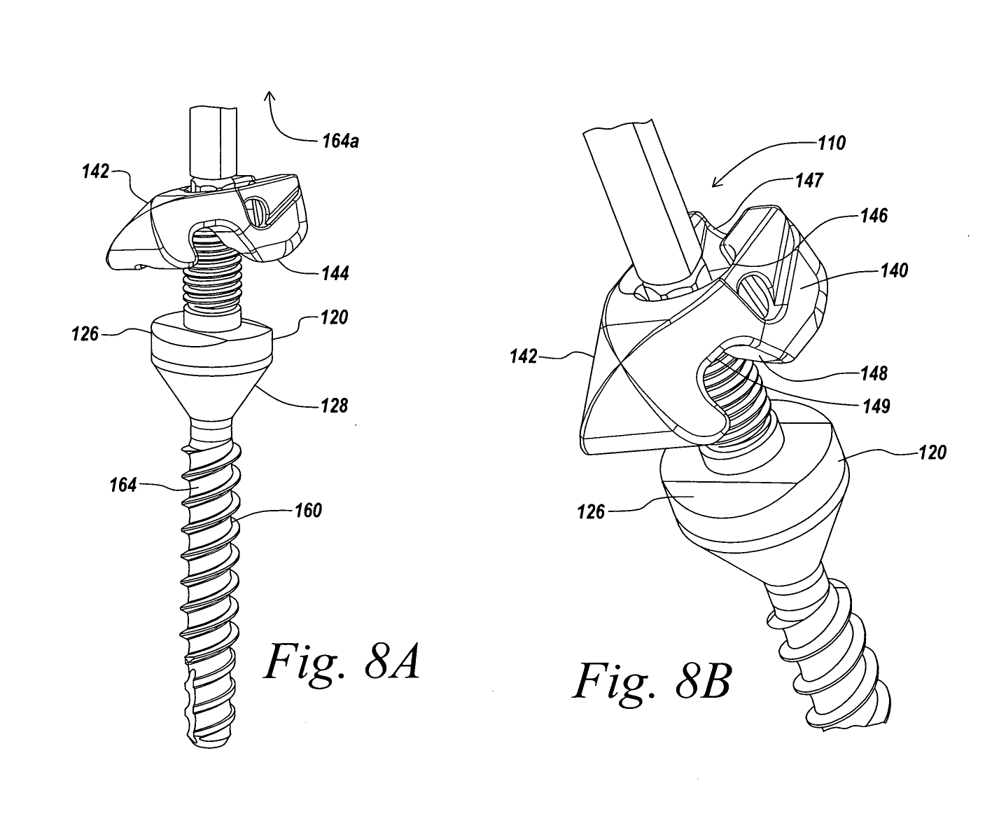

The seat element 120 may adapt to seat the rod 190 whose rod axis 192 is out-of-plane relative to the bone anchor axis 162 by deforming to match the out-of-plane orientation of the rod 190. As depicted in FIG. 8A, the seat element 120 may have a deformable portion 126 formed of a sufficiently compliant material to conform to a surface of the elongate spinal fixation element 190. The sufficiently compliant material may be semi-deformable or deformable. The sufficiently compliant material may undergo reversible or irreversible deformation when the deformable portion 126 conforms to a surface of the elongate spinal fixation element 190. A sufficiently compliant material may be a type of polymer, for example a silicone, a polyurethane, a polyethylene, a polyvinyl, a polyether ether ketone (PEEK), etc., or a combination of polymers a composite material, or any other sufficiently compliant and bio-compatible material.

The deformable portion 126 disposed on a top side 122 of the seat element 120 may cover only a portion of the top side, as depicted in FIGS. 8A and 8B, or the deformable portion 126 may cover all of the top side 122 of the seat element 120. The deformable portion 126 may be affixed to the rest of the seat element 120 by an interference fit. The deformable portion 126 of the seat element 120 may be affixed to the shaft 164, or formed in one piece with the shaft 164, using a technique like overmolding. The deformable portion 126 may be attached to the rest of the seat element 120 using any other means known in the art.

As shown in FIG. 8A, a non-deformable portion of the seat element 120 may be formed in one piece with shaft 164 of the bone anchor 60 and may include a flared portion 128 that flares in a direction of a proximal end of the shaft 164a. The seat element 120, including the flared portion 128, may be solidly affixed to the bone anchor 160, or the seat element 20 may be free to rotate around a shaft 164 of the bone anchor 160, according to aspects of the present invention. When the bone anchor 160 is inserted adjacent to the rod 190 the flared portion 128 of the seat element 120 contacts the elongate spinal fixation element 190 and deflects rod 190 away from the bone anchor 160. The shape of the flared portion 128 allows the seat element 120 to slide past the rod 190, displacing the rod 190 away from the bone anchor 160, during insertion without becoming caught on the rod 190. When the bone anchor 160 is inserted sufficiently deep that top side 122 of the seat element 120 inserted deeper in the bone than the rod 190, then the rod 190 “snaps” back to its undisplaced position producing tactile and/or auditory feedback to a surgeon indicating that the elongate spinal fixation element 190 is properly seated, as described in detail previously with reference to FIGS. 6A to 6G. As well as adjusting to the orientation of the rod axis 192, the semi-deformable portion 126 of the seat element 120 reduces the transmission of vibrations between the rod 190 and the bone anchor 160 by acting as a damper at the contact between the rod 190 and the seat element 120.

In the embodiment depicted in FIGS. 1A-4 and FIGS. 6A-6G, the seat element 20 adapts to the out-of-plane orientation of the elongate spinal fixation element axis 192 by pivoting. In the embodiment depicted in FIGS. 7-8B the seat element 120 adjusts to the orientation of the rod axis 192 by deforming. Other embodiments that include a seat element that adapts to an out-of-plane orientation of an elongate spinal fixation element by both pivoting and deforming also fall within the scope of the present invention.

According to aspects of the present invention, the clamp element 140 of the adaptable clamping mechanism 110 may adapt to the out-of-plane orientation of the elongate spinal fixation element axis 192 by pivoting and may include a deformable portion 149. The clamp element 140 may have a top side 142 and a bottom side 144 facing the seat element 120 when in use. The clamp element 140 may include a channel 146 for passing the shaft 164 of the bone anchor 160 through. The clamp element 140 may also include a trough recess 148 in the bottom side 144 of the clamp element 140 configured to contact the rod 190. The deformable portion 149 may be disposed at a surface of the trough recess 148 where the clamp element 140 contacts the elongate spinal fixation element 190. A width of the channel 146 in the clamp element 140 may be larger in one or more directions than in other directions allowing the clamp element to pivot further in some directions than in others, as depicted in FIG. 8B. The deformable portion 149 of the clamp element 140 may damp vibrations transmitted through the rod 190 to the bone anchor, as well as aiding in coupling the clamp element 140 with the rod 190.

When a securing element (not shown) is tightened against the top surface 142 of the clamp element 140, pressure from the securing element on the clamp element 140 causes the clamp element 140 to pivot and causes the deformable portion 149 of the clamp element to conform to a shape of the rod 190. The deformable portion 149 of the clamp element 140 creates a high coefficient of friction between the clamp element 140 and the elongate spinal fixation element 190, and the semi-deformable portion 149 reduces transmission of vibrations between the elongate spinal fixation element 190 and the patient's bone by dampening a contact between the clamp element 140 and the rod 190. Pressure from the securing element is transmitted through the clamp element 140 which exerts pressure on the rod 190. The pressure on the rod 190 is transmitted thorough the rod 190 to the seat element 120 causing a deformable portion 126 of the seat element 120 to deform. The deformable portion 126 of the seat element and the deformable portion 149 of the clamp element allow the adaptable clamping mechanism 110 to adapt to clamp a rod 190 with a significant longitudinal curvature as well as allowing the adaptable clamping mechanism 110 to adapt to clamp an elongate spinal fixation element 190 with an out-of-plane orientation with respect to the bone anchor 160.

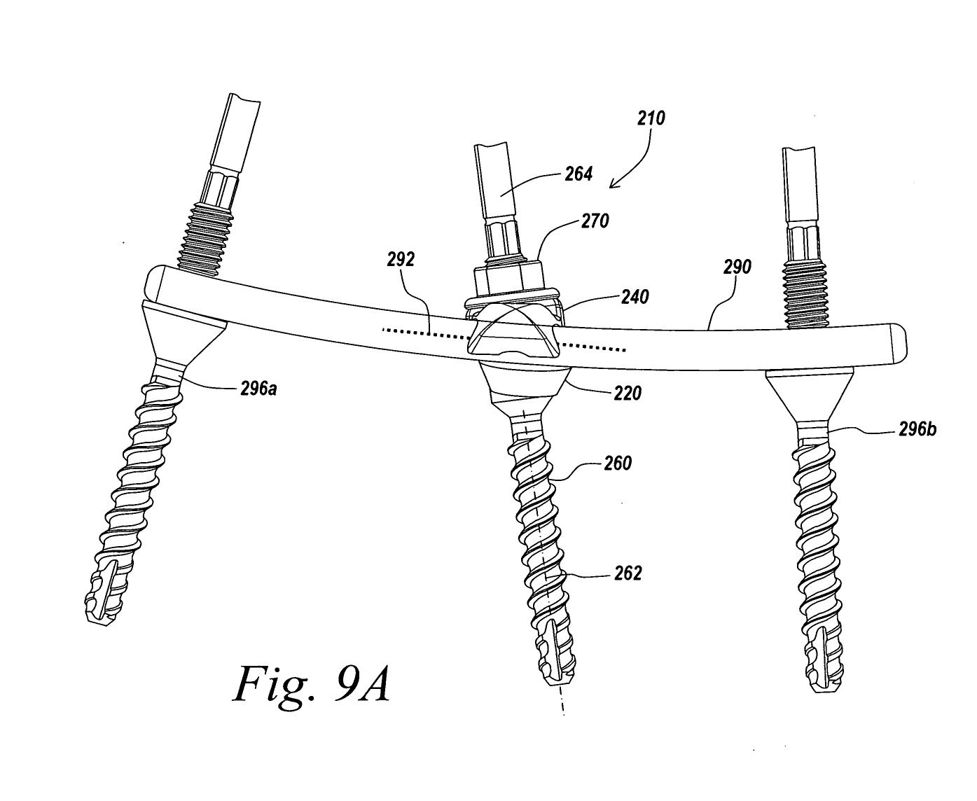

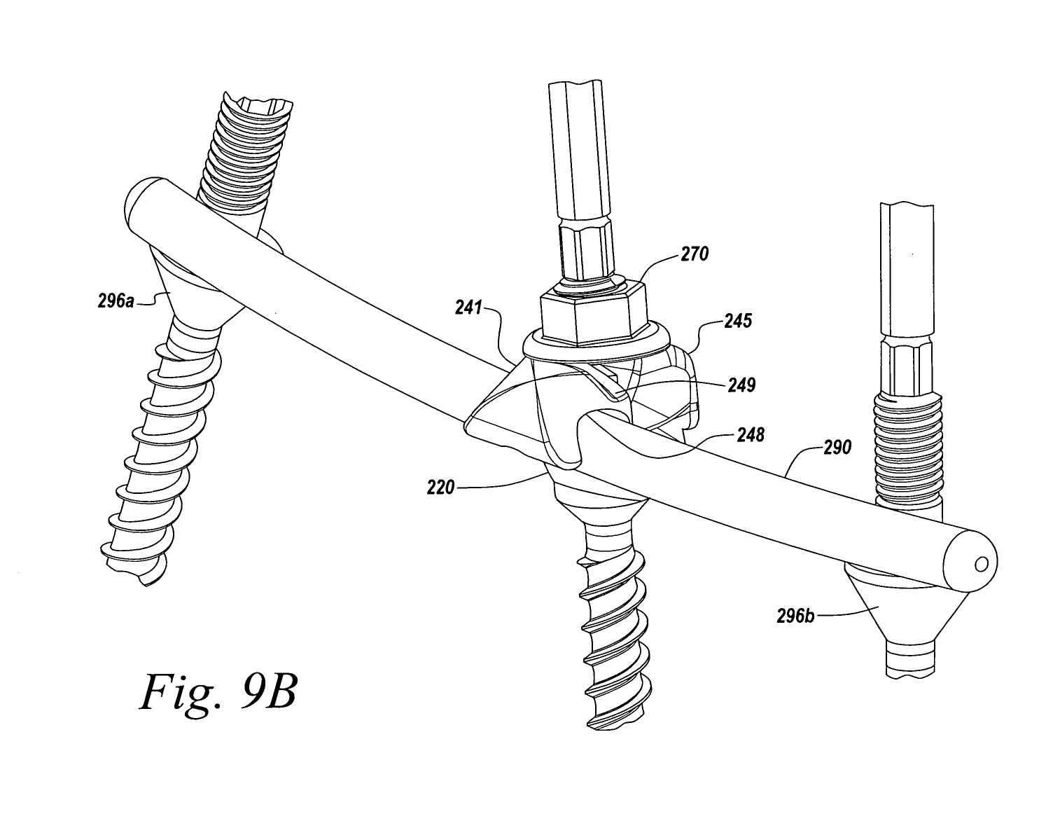

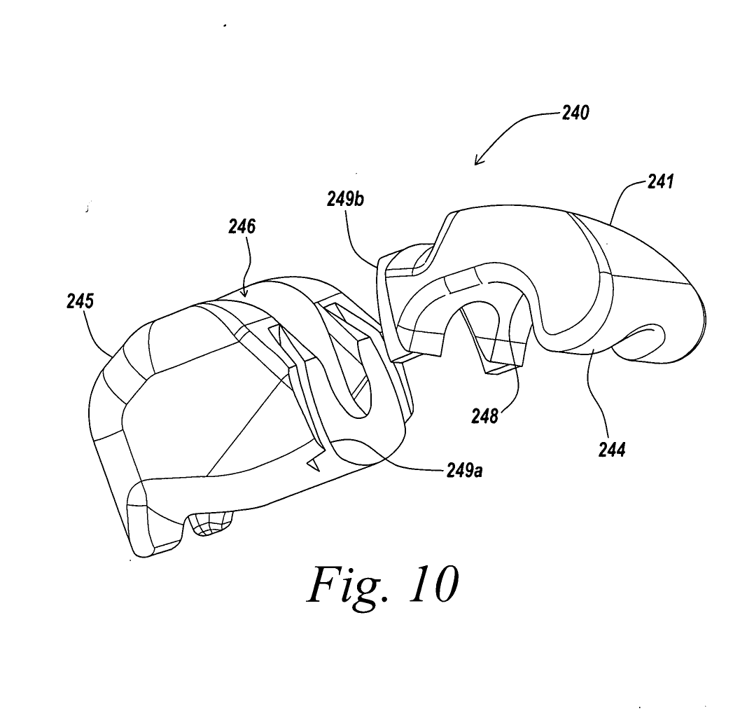

FIGS. 9A, 9B and 10 illustrate another exemplary embodiment of the adaptable clamping mechanism 210 for coupling an elongate spinal fixation element 290, to a bone anchor 260. A clamp element 240 of the adaptable clamping mechanism 210 may include a portion configured to rotate about an axis perpendicular to a central axis 262 of the bone anchor 260, according to aspects of the present invention. FIGS. 9A and 9B depict the adaptable clamping mechanism 210 in use securing an elongate spinal fixation element 290 whose elongate spinal fixation element axis 292 is non-perpendicular relative to the central axis 262 of the bone anchor 260. Also depicted are two bone anchors 296a, 296b with non-adaptable seating elements supporting opposite ends of the elongate spinal fixation element 290. The seat element depicted 220 is a polyaxial seat element as described previously (see description of FIGS. 1A-4).

The seat element 220 of the adaptable clamping mechanism 210 adapts to an orientation of the elongate spinal fixation element axis 292 by pivoting. The clamp element 240 of the adaptable clamping mechanism 210 adapts to an orientation of the elongate spinal fixation element axis 292 by a portion of the clamp element 240 rotating about an axis perpendicular to the bone anchor 260, while another portion of the clamp element 240 remains aligned with the bone anchor 260.

The clamp element 240 may include an attachment element 245 that secures the clamp element 240 to the shaft 264 of the bone anchor 260, and a rotating element 241 that contacts the elongate spinal fixation element 290 and is rotatably coupled with the attachment element 245. The rotating element 241 is configured to clamp the elongate spinal fixation element 290, against the seat element 220. The rotating element 241 is also configured to rotate relative to the attachment element 245 about an axis perpendicular to the bone anchor axis 262. The attachment element 245 and the rotating element 241 may be rotatably coupled with a sliding dovetail joint 249.

FIG. 10 depicts an exploded view of the clamp element 240. The attachment element 245 has a channel 246 for a shaft 264 of the bone anchor 260 to pass through. The rotating element 241 has a bottom side 244 facing the seat element 220, and includes a trough recess 248 formed in the bottom side 244 and configured to contact a surface of the elongate spinal fixation element 290. The path of the sliding dovetail joint 249a, 249b forms a portion of an arc allowing the attachment element 245 and the rotating element 241 to rotate relative to each other.

Although exemplary embodiments of the adaptable clamping mechanism 10, 110, 210 are described as combinations of a particular seat element and a particular clamp element, one of skill in the art will recognize that the seat elements, clamp elements and any other elements or features of the adaptable clamping mechanism can be used in combinations not specifically recited in this description.

Embodiments of the adaptable clamping mechanisms may be constructed of any biocompatible material including, for example, metals, such as stainless steel or titanium, polymers, ceramics, or composites thereof. The size and diameter of elements of the adaptable clamping mechanism may vary depending on many factors including: the type of bone anchor used, the type of elongate spinal fixation element used, the diameter of a surgical access port or minimally invasive incision for insertion of the bone anchor and adaptable clamping mechanism, etc.

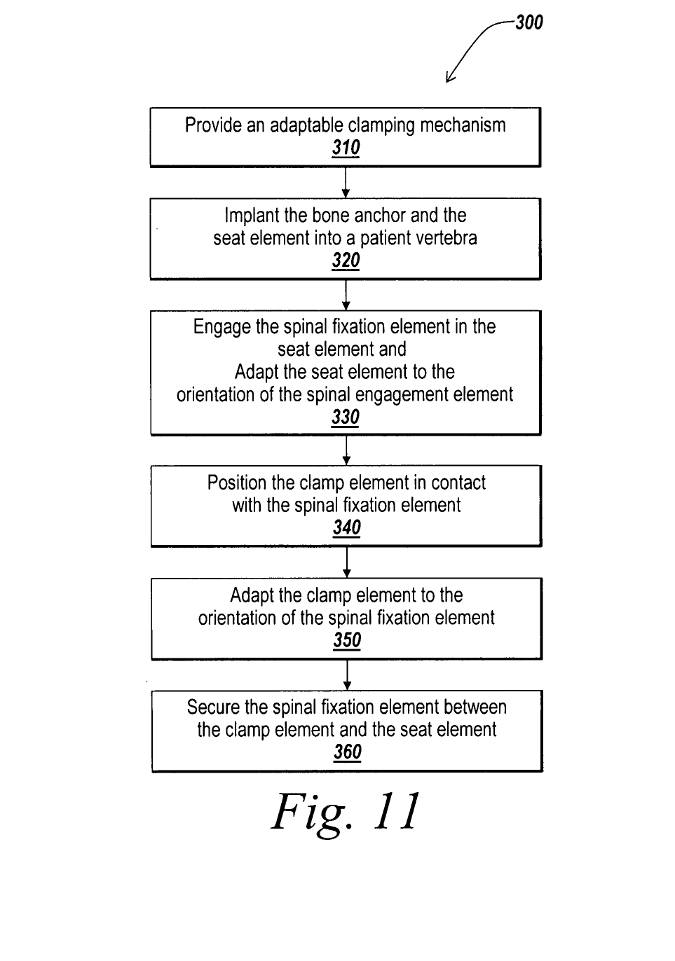

FIG. 11 illustrates an exemplary embodiment of the present invention that provides a method 300 for coupling an elongate spinal fixation element that has been positioned to extend along a patient's spinal column to a vertebra of the patient using an adaptable clamping mechanism. Solely for illustrative purposes, the method will be generally described with respect to the adaptable clamping mechanism 10, elongate spinal fixation element 90 and bone anchor 60 depicted in FIGS. 1A-4.

First, an adaptable clamping mechanism 10 is provided (step 310). The adaptable clamping mechanism 10 includes a seat element 20 disposed on the bone anchor 60 and configured to adapt to seat a elongate spinal fixation element 90 with a axis 92 that is non-perpendicular relative to a central axis 62 of the bone anchor 60. The adaptable clamping mechanism 10 also includes a clamp element 40 configured to adapt to hold the elongate spinal fixation element 90 against the seat element 20. The adaptable clamp mechanism 10 is configured for substantially side engagement with the elongate spinal fixation element 90, meaning that the seat element 20 is configured to receive the elongate spinal fixation element 90 in an engagement direction that is perpendicular to the longitudinal axis 92 of the elongate spinal fixation element 90 and toward the central axis 62 of the bone anchor 60. The adaptable clamping mechanism 10 may also include a securing element 70.

The bone anchor 60 and the attached seat element 20 of the adaptable clamping mechanism 10 are implanted into a vertebra of the patient (step 320). The elongate spinal fixation element 90 is engaged in the seat element 20 from the side and the seat element 20 is adapted to an orientation of the elongate spinal fixation element 90 (step 330). The clamp element 40 is positioned on the shaft 64 of the bone anchor 60 in contact with the elongate spinal fixation element 90 (step 340). The clamp element 40 is adapted to an orientation of the elongate spinal fixation element 90 (step 350). The elongate spinal fixation element 90 is secured between the seat element and the clamp element 40 (step 360). The clamp element 40 may include a securing mechanism or a separate securing element 70 may be used to secure the clamp element 40 against the elongate spinal fixation element 90.

According to aspects of the present invention, the method may include including inserting and positioning an elongate spinal fixation element 90 in a patient through a minimally invasive surgical access port before implanting the bone anchor 60 and the seat element 20 into the patient's vertebra. The bone anchor 60 and the seat element 20 may be implanted into a patient's vertebra through a minimally invasive surgical access port. The clamp element 40 may be positioned in contact with the elongate spinal fixation element 90 through a minimally invasive surgical access port.

According to other aspects of the present invention, the method may also include removing the breakaway portion 76 of the shaft 64. Implanting a bone anchor 60 and the attached seat element 20 of the adaptable clamping mechanism 10 may include making physical contact between the seat element 20 and the elongate spinal fixation element 90 during insertion of the bone anchor 60 producing feedback to a surgeon regarding a position of the seat element 20 relative to the elongate spinal fixation element 90.

According to additional aspects of the present invention, adapting the seat element 20 to an orientation of the elongate spinal fixation element 90 may include pivoting the seat element 20 relative to the bone anchor 60. Adapting the seat element 120 to an orientation of the elongate spinal fixation element 190 may include deforming a portion of the seat element 120 to conform to a surface of the elongate spinal fixation element 190. Adapting the clamp element 40 to an orientation of the elongate spinal fixation element 90 may include pivoting the clamp element 40 relative to the bone anchor 60. Adapting the clamp element 240 to an orientation of the elongate spinal fixation element 290 may include rotating a portion of the clamp element 240 about an axis perpendicular to the central axis 262 of the bone anchor 260. Adapting the clamp element 140 to an orientation of the elongate spinal fixation element 190 may include deforming a portion of the clamp element 140 to conform to a surface of the elongate spinal fixation element 190

Although the adaptable clamping mechanism 10 is depicted in the figures in conjunction with a particular bone anchor 60, and with a particular elongate spinal fixation element 90, one of ordinary skill in the art will recognize that embodiments of an adaptable clamping mechanism configured to couple any one of many different types of bone anchor to any one of many different types of elongate spinal fixation element fall within the scope of the present invention.

Although exemplary embodiments of the present invention depict coupling a particular elongate spinal fixation element, a spinal rod, to a particular bone anchor, a pedicle screw, one of ordinary skill in the art will recognize that that embodiments of the present invention are not limited to use with pedicle screws and spinal rods. Embodiments of the present invention may be used with different types of elongate spinal fixation elements including, but not limited to: plates, PDS (posterior dynamic stabilization) devices, solid rods, dynamic rods, etc. Embodiments of the present invention may be used with different types of bone anchors including, but not limited to: facet screws, bolts, staples, anchors, etc.

While the mechanisms and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. An adaptable clamping mechanism for coupling an elongate spinal fixation element to a bone anchor, the adaptable clamp mechanism comprising:
a seat element disposed on the bone anchor and configured to adapt to seat the elongate spinal fixation element whose longitudinal axis is non-perpendicular with respect to a central axis of the bone anchor, the seat element also configured to receive the elongate spinal fixation element in an engagement direction that is perpendicular to the longitudinal axis of the elongate spinal fixation element and toward the central axis of the bone anchor; and
a clamp element configured to adapt to hold the elongate spinal fixation element against the seat element.

2. The adaptable clamping mechanism of claim 1, further comprising a securing element configured to secure the clamp element against the elongate spinal fixation element.

3. The adaptable clamping mechanism of claim 1, wherein the seat element and the clamp element are configured to adapt over a predetermined total minimum angular range of at least about 50 degrees.

4. The adaptable clamping mechanism of claim 1, wherein the seat element is configured to pivot relative to the central axis of the bone anchor in at least one direction.

5. The adaptable clamping mechanism of claim 4, the seat element comprising:
a pivotable contact disposed on a bottom side of the seat element configured to contact a shaft of the bone anchor in a pivotable fashion;
a top side of the seat element configured to seat an elongate spinal fixation element; and
a channel allowing a shaft of the bone anchor to pass through the seat element, wherein a channel diameter is significantly larger than a shaft diameter to allow the seat element to pivot relative to the shaft.

6. The adaptable clamping mechanism of claim 1, wherein a portion of the seat element is formed of a sufficiently compliant material to conform to a surface of the elongate spinal fixation element.

7. The adaptable clamping mechanism of claim I, wherein the clamp element is configured to pivot relative to the central axis of the bone anchor in at least one direction.

8. The adaptable clamping mechanism of claim 7, the clamp element having:
a top side and a bottom side;
a channel through the clamp element for passing a shaft of the bone anchor through, and wherein a channel diameter is sufficiently larger than a shaft diameter to allow the clamp to pivot relative to the shaft; and
a trough recess in the bottom side of the clamp element configured to contact the elongate spinal fixation element.

9. The adaptable clamping mechanism of claim I, the clamp element comprising:
an attachment element having a channel for a shaft of the bone anchor to pass through; and
a rotating element rotatably coupled with the attachment element and configured to clamp the elongate spinal fixation element against the seat element, wherein the rotating element is configured to rotate relative to the attachment element about an axis perpendicular to the central axis of the bone anchor.

10. The adaptable clamping mechanism of claim 1, wherein a portion of the clamp element is formed of a sufficiently compliant material to conform to a surface of the elongate spinal fixation element.

11. The adaptable clamping mechanism of claim 1, wherein the seat element and the clamp element are sized and dimensioned to be inserted into a patient through a minimally invasive surgery access port.

12. The clamping mechanism of claim 1, wherein the seat element is configured to provide feedback regarding a position of the elongate spinal fixation element relative to a position of the seat element.

13. A bone anchor assembly for securing a spinal fixation element, the bone anchor assembly comprising:
a bone anchor with a central axis;
a seat element disposed on the bone anchor and configured to adapt to seat the elongate spinal fixation element whose longitudinal axis is non-perpendicular with respect to the central axis of the bone anchor, the seat element also configured to receive the elongate spinal fixation element in an engagement direction that is perpendicular to the longitudinal axis of the elongate spinal fixation element and toward the central axis of the bone anchor;
a clamp element configured to adapt to hold the elongate spinal fixation element against the seat element; and
a securing element configured to secure the clamp element against the spinal fixation element.

14. A method of coupling an elongate spinal fixation element positioned to extend along a patient's spinal column to a bone anchor implanted in the patient's vertebra, the method comprising:
providing an adaptable clamping mechanism, comprising:
a seat element disposed on the bone anchor and configured to adapt to seat an elongate spinal fixation element with a longitudinal axis that is non-perpendicular relative to the central axis of the bone anchor, the seat element also configured to receive the elongate spinal fixation element in an engagement direction that is perpendicular to the longitudinal axis of the elongate spinal fixation element and toward the central axis of the bone anchor; and
a clamp element configured to adapt to hold the elongate spinal fixation element against the seat element;
implanting the bone anchor having a shaft and the attached seat element of the adaptable clamping mechanism into one of the patient's vertebra,
engaging the elongate spinal fixation element in the seat element by reducing a separation between the spinal fixation element and the central axis of the bone anchor and adapting the seat element to an orientation of the elongate spinal fixation element;
positioning the clamp element of the adaptable clamping mechanism on the shaft in contact with the elongate spinal fixation element;
adapting the clamp element to an orientation of the elongate spinal fixation element; and
securing the elongate spinal fixation element between the clamp element and the seat element.

15. The method of claim 14, wherein the method is repeated for coupling the elongate spinal fixation element to an additional bone anchor inserted in one of the patient's vertebrae.

16. The method of claim 14, wherein implanting a bone anchor having a shaft and the attached seat element of the adaptable clamping mechanism includes implanting the bone anchor adjacent to the elongate spinal fixation element and making physical contact between the seat element and the elongate spinal fixation element during implantation of the bone anchor producing feedback to a surgeon regarding a position of the seat element relative to the elongate spinal fixation element.

17. The method of claim 14, wherein adapting the seat element to an orientation of the elongate spinal fixation element includes pivoting the seat element relative to the bone anchor.

18. The method of claim 14, wherein adapting the seat element to an orientation of the elongate spinal fixation element includes deforming a portion of the seat element to conform to a surface of the elongate spinal fixation element.

19. The method of claim 14, wherein adapting the clamp element to an orientation of the elongate spinal fixation element includes pivoting the seat element relative to the bone anchor.

20. The method of claim 14, wherein adapting the clamp element to an orientation of the elongate spinal fixation element includes rotating a portion of the clamp element about an axis perpendicular to the central axis of the bone anchor.

21. The method of claim 14 wherein adapting the clamp element to an orientation of the elongate spinal fixation element includes deforming a portion of the clamp to conform to a surface of the spinal fixation element.
